(19) United States
(12) Patent Application Publication
(10) Pub. No.: US 2009/0054736 A1
(43) Pub. Date: Feb. 26, 2009

(54) METHOD AND SYSTEM FOR A PATIENT MONITORING ALARM

(75) Inventors: Borje Rantala, Helsinki (FI); Leena Pesu, Helsinki (FI)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/843,891

(22) Filed: Aug. 23, 2007

(51) Int. Cl. A61B 5/00

(52) U.S. Cl. 600/300

(57) ABSTRACT

A method for implementing a patient monitoring system having an alarm is disclosed herein. The method includes providing a plurality of triggering criteria, and overriding a category of the plurality of triggering criteria if an override signal has been received from an input device. The process of overriding a category of triggering criteria is adapted to minimize false alarms. The method for implementing a patient monitoring system having an alarm also includes sounding an alarm if one of the triggering criteria has been met and has not been overridden. A corresponding patient monitoring system is also provided.

### BACKGROUND OF THE INVENTION

Patient monitoring systems commonly include an alarm adapted to automatically alert hospital personnel in the event of an emergency. The triggering criteria for such an alarm is generally selectable. According to one exemplary embodiment, the patient monitoring system collects patient data such as, for example, the patient's vital signs, and the alarm is activated if any collected patient data falls outside a predefined range. In this manner, the process of monitoring the patient is automated thereby minimizing the requisite patient care burden. Additionally, the level of care afforded each patient is not appreciably diminished because the patient monitoring alarm ensures that patients receive direct supervision at the most appropriate time.

One problem with conventional alarm systems is that they may be unintentionally activated by patient motion or manipulation. It has been observed that during the process of administering care, hospital personnel can move the patient enough to inadvertently activate the alarm. When a false alarm sounds, other hospital personnel may have to stop what they are doing in order to ascertain the cause of the alarm thereby needlessly wasting valuable resources. False alarms may also create an unnecessary distraction or disruption and are therefore undesirable.

### BRIEF DESCRIPTION OF THE INVENTION

The above-mentioned shortcomings, disadvantages and problems are addressed herein which will be understood by reading and understanding the following specification.

In an embodiment, a method for implementing a patient monitoring system having an alarm includes providing a plurality of triggering criteria, and overriding a category of the plurality of triggering criteria if an override signal has been received from an input device. The process of overriding a category of triggering criteria is adapted to minimize false alarms. The method for implementing a patient monitoring system having an alarm also includes sounding an alarm if one of the triggering criteria has been met and has not been overridden.

In another embodiment, a method for implementing a patient monitoring system having an alarm includes providing patient data from a plurality of sensors, providing a plurality of triggering criteria based on the patient data, and overriding a category of the triggering criteria if an override signal has been received from an input device. The category of the triggering criteria includes one or more triggering criteria that are potentially related to patient manipulation. The process of overriding a category of triggering criteria is adapted to minimize false alarms. The method for implementing a patient monitoring system having an alarm also includes sounding an alarm if one of the triggering criteria has been met and has not been overridden.

In another embodiment, a patient monitoring system includes an input device adapted to selectively transmit an override signal, and a controller coupled with the input device. The controller is adapted to evaluate a plurality of triggering criteria, and is configured to override a category of the triggering criteria in response to the receipt of the override signal from the input device. The patient monitoring system also includes an alarm coupled with the controller. The controller is configured to activate the alarm if one of the triggering criteria has been met and has not been overridden.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a patient monitoring system attached to a patient in accordance with an embodiment; and

FIG. 2 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

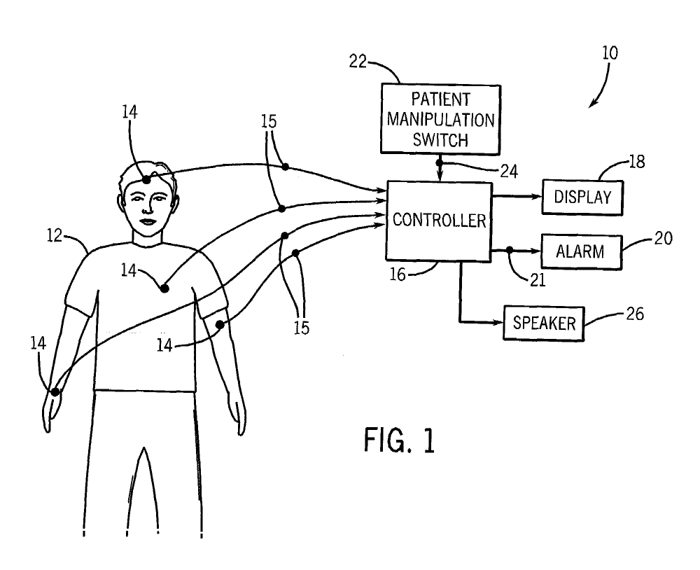

Referring to FIG. 1, a patient monitoring system 10 attached to a patient 12 is shown in accordance with an embodiment. The patient monitoring system 10 includes a plurality of sensors 14, a controller 16, a display 18, an alarm 20, a patient manipulation switch 22, and/or a speaker 26. The sensors 14 may, for example, include conventional leads, probes, or any other known transducer devices. In a non-limiting manner, the sensors 14 may include a device adapted to monitor one or more patient vital signs such as body temperature, pulse rate, heart rate, blood pressure and respiratory rate.

The controller 16 is configured to receive sensor signals 15 from the sensors 14, process the sensor signals 15 in order to produce patient data, and transmit the patient data to the display 18. The display 18 conveys the patient data from the controller 16 in a convenient form. The controller 16 is also configured to evaluate the patient data in a known manner, and to send an alarm signal 21 adapted to activate the alarm 20 in response to one or more triggering criteria. For purposes of this disclosure, the term “triggering criteria” is defined to include any specified conditions under which the alarm 20 is to be activated or triggered. Triggering criteria are generally predefined and are selectable to meet the needs of a given patient. As an example, a first triggering criterion may be defined by a range of electrocardiogram (ECG) data, a second triggering criterion may be defined by a range of blood pressure data, and a third triggering criterion may be defined by a range of thermal data. Accordingly, the alarm 20 can be automatically activated in order to alert hospital personnel if, for example, any one of a given patient's vital signs are excessively high or low.

The patient manipulation switch 22 is configured to send an override signal 24 to the controller 16 such that the controller 16 can override one or more triggering criteria for a selectable amount of time (e.g., 2-5 minutes). For purposes of this disclosure, the patient manipulation switch 22 should be defined to include any input device adapted to selectively transmit an electrical signal. It should be appreciated that the alarm 20 cannot be activated based on an overridden triggering criterion. The selectable amount of time during which the triggering criteria are overridden is generally adapted to allow a caregiver sufficient time to attend to or otherwise manipulate the patient 12 without triggering a false alarm. In a non-limiting manner, false alarms may include alarms that are triggered by events unrelated to the condition of the patient 12 and/or alarms that are unintentionally triggered by patient manipulation. The controller 16 may be configured such that, when the patient manipulation switch 22 is activated twice in close succession, all triggering criteria are enabled. Therefore, if the patient manipulation switch 22 is inadvertently activated, it can be activated a second time in order to avoid unnecessarily overriding any triggering criteria.

According to one embodiment, the controller 16 is configured to override only a predefined category of triggering criteria in response to an override signal 24 from the patient manipulation switch 22. As an example, the predefined category of triggering criteria to be overridden may include any criteria that are potentially related to the manipulation of the patient 12 such as criteria based on motion artifacts, or on the disconnection of leads and probes. Therefore, any triggering criteria unrelated to patient manipulation remain operational so that appropriate personnel can still be alerted to an emergency situation that occurs while the patient 12 is being treated or cared for.

The patient manipulation switch 22 can be positioned near the patient 12 such as, for example, in close proximity to the patient's bed (not shown) such that hospital personnel can conveniently activate the patient manipulation switch 22 before attending to the patient 12. The patient manipulation switch 22 can be indirectly connected to the display 18 via the controller 16 so that hospital personnel are provided with a visual indication of triggering criteria status. Similarly, the patient manipulation switch 22 can be indirectly connected to the speaker 26 via the controller 16 so that hospital personnel are provided with an audible indication of triggering criteria status. For purposes of this disclosure, the term “triggering criteria status” identifies whether a group of triggering criteria are either activated or overridden.

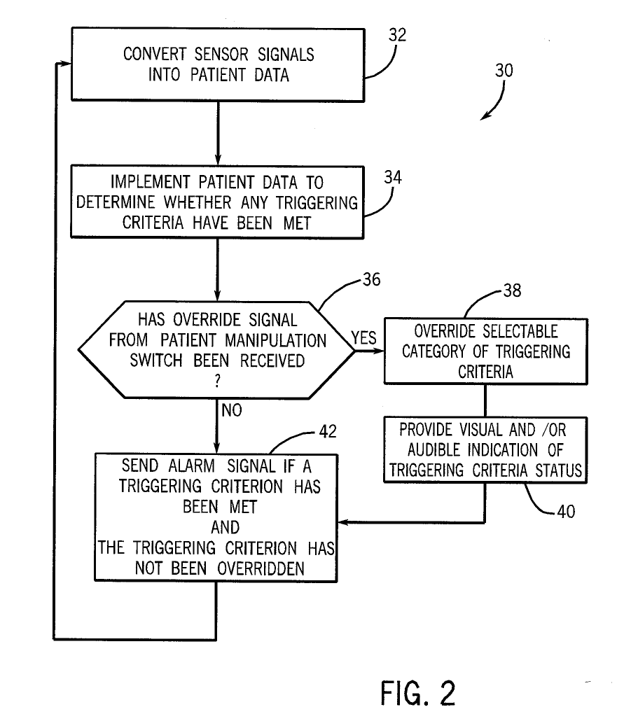

FIG. 2 is flow chart illustrating a method 30 shown according to one embodiment. The individual blocks 32-42 of the flow chart represent steps that may be performed in accordance with the method 30. According to an embodiment, the method 30 may be stored as an algorithm on a memory device (not shown) included as part of the controller 16 (shown in FIG. 1).

Referring now to FIGS. 1 and 2, at step 32, sensor signals 15 from the sensors 14 are converted to patient data. At step 34, the patient data of step 34 is implemented to determine whether any triggering criteria have been met. At step 36, the method 30 determines whether an override signal 24 has been received from the patient manipulation switch 22. If at step 36 an override signal 24 has been received, the method proceeds to step 38. If at step 36 an override signal 24 has not been received, the method proceeds to step 42.

At step 38, the method 30 overrides one or more categories of triggering criteria for a predetermined amount of time. The overridden categories are generally selectable but may alternatively be fixed. According to one embodiment, a category of triggering criteria related to patient manipulation are overridden for a period of 2-5 minutes. According to another embodiment, all triggering criteria are overridden for a first time period (e.g., 1 minute), and thereafter only the triggering criteria related to patient manipulation are overridden for a second time period (e.g., 2-5 minutes).

Step 40 is an optional step wherein the method 30 provides a visual and/or audible indication of triggering criteria status. According to one embodiment, the visual indication is in the form of a graphic depicted on the display 18. The graphic may include both a visual indication that one or more triggering criteria are being overridden, and a timer showing the amount of time remaining until the triggering criteria are reactivated.

At step 42, an alarm signal 21 is sent to the alarm 20 if at least one triggering criterion has been met and has not been overridden. The alarm signal 21 is configured to sound the alarm 20 in order to alert appropriate hospital personnel. After completing step 42, the method 30 returns to step 32.

While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. A method for implementing a patient monitoring system having an alarm comprising:
providing a plurality of triggering criteria;
overriding a category of the plurality of triggering criteria if an override signal has been received from an input device, wherein said overriding a category of the plurality of triggering criteria is adapted to minimize false alarms; and
sounding an alarm if one of the plurality of triggering criteria has been met and said one of the plurality of triggering criteria has not been overridden.

2. The method of claim 1, further comprising providing a visual indication of the status of one or more of the plurality of triggering criteria.

3. The method of claim 1, further comprising providing an audible indication of the status of one or more of the plurality of triggering criteria.

4. The method of claim 1, wherein said overriding a category of the plurality of triggering criteria includes overriding one or more of the plurality of triggering criteria that are related to the manipulation of a patient.

5. The method of claim 1, wherein said overriding a category of the plurality of triggering criteria includes overriding one or more of the plurality of triggering criteria that are related to a sensor motion.

6. The method of claim 1, wherein said overriding a category of the plurality of triggering criteria includes overriding one or more of the plurality of triggering criteria that are related to a disconnection of a sensor from a patient.

7. The method of claim 1, wherein said overriding a category of the plurality of triggering criteria includes overriding a category of the plurality of triggering criteria for a predetermined amount of time.

8. A method for implementing a patient monitoring system having an alarm comprising:
providing patient data from a plurality of sensors;
providing a plurality of triggering criteria based on said patient data;
overriding a category of the plurality of triggering criteria if an override signal has been received from an input device, the category of the triggering criteria being defined to include one or more of the plurality of triggering criteria that are potentially related to patient manipulation, wherein said overriding a category of the plurality of triggering criteria is adapted to minimize false alarms; and
sounding an alarm if one of the plurality of triggering criteria has been met and said one of the plurality of triggering criteria has not been overridden.

9. The method of claim 8, further comprising providing a visual indication of the status of one or more of the plurality of triggering criteria.

10. The method of claim 8, further comprising providing an audible indication of the status of one or more of the plurality of triggering criteria.

11. The method of claim 8, wherein said overriding a category of the plurality of triggering criteria includes overriding one or more of the plurality of triggering criteria that are related to a sensor motion.

12. The method of claim 8, wherein said overriding a category of the plurality of triggering criteria includes overriding one or more of the plurality of triggering criteria that are related to a disconnection of a sensor from a patient.

13. The method of claim 8, wherein said overriding a category of the plurality of triggering criteria includes overriding a category of the plurality of triggering criteria for a predetermined amount of time.

14. A patient monitoring system comprising:
an input device adapted to selectively transmit an override signal;
a controller coupled with the input device, the controller being adapted to evaluate a plurality of triggering criteria, the controller being configured to override a category of the plurality of triggering criteria in response to the receipt of the override signal from the input device; and
an alarm coupled with the controller;
wherein the controller is configured to activate the alarm if one of the plurality of triggering criteria has been met and said one of the plurality of triggering criteria has not been overridden.

15. The patient monitoring system of claim 14, wherein the input device includes a patient manipulation switch.

16. The patient monitoring system of claim 14, further comprising a display configured to visually convey the status of one or more of the plurality of triggering criteria.

17. The patient monitoring system of claim 14, further comprising a speaker configured to audibly convey the status of one or more of the plurality of triggering criteria.

18. The patient monitoring system of claim 14, further comprising a plurality of sensors coupled with the controller and operatively connected to a patient.
